(19) United States
(12) Patent Application Publication
(10) Pub. No.: US 2025/0043365 A1
(43) Pub. Date: Feb. 6, 2025

(54) Adenosine triphosphatase sarcoplasmic/endoplasmic reticulum Ca2+ transporting 2 (ATP2A2) gene-based molecular marker for identifying chicken feed efficiency trait, and identification method and use thereof

(71) Applicant: Anhui Agricultural University, Hefei City (CN)

(72) Inventors: Sihua JIN, Hefei City (CN); Yuanfei DING, Hefei City (CN); Zhaoyu GENG, Hefei City (CN); Yi FANG, Hefei City (CN); Xin WANG, Hefei City (CN); Xiaoqing HU, Hefei City (CN); Xin WANG, I, Hefei City (CN); Xuling LIU, Hefei City (CN); Xing LIU, Hefei City (CN); Yiran LUO, Hefei City (CN); Lu FENG, Hefei City (CN); Dehua ZHA, Hefei City (CN)

(21) Appl. No.: 18/672,154

(22) Filed: May 23, 2024

(30) Foreign Application Priority Data
May 23, 2023 (CN) 202310596686.4

(51) Int. Cl. C12Q 1/6888; C12Q 1/44; C12Q 1/6806; C12Q 1/686; G01N 27/447

(52) U.S. Cl. CPC C12Q 1/6888; C12Q 1/44; C12Q 1/6806; C12Q 1/686; G01N 27/44726; C12Q 2600/124; G01N 2333/916

(57) ABSTRACT

Provided are an adenosine triphosphatase sarcoplasmic/endoplasmic reticulum Ca²⁺ transporting 2 (ATP2A2) gene-based molecular marker for identifying a chicken feed efficiency trait, and an identification method and use thereof. A nucleotide sequence of the molecular marker is shown in SEQ ID NO: 1, and a base at locus 926 in the nucleotide sequence is T or G. In the present application, by identifying a genotype of the molecular marker in a chicken genome and then selecting the chicken feed efficiency trait according to the genotype, a breeding method for early selection of the poultry feed efficiency trait is established. The method is simple, rapid, and low-cost, does not require special instruments, and is suitable for the needs of marker-assisted selection experiments.

### CROSS-REFERENCE TO RELATED APPLICATION

This patent application claims the benefit and priority of Chinese Patent Application No. 202310596686.4 filed with the China National Intellectual Property Administration on May 23, 2023, the disclosure of which is incorporated by reference herein in its entirety as part of the present application.

### REFERENCE TO SEQUENCE LISTING

A computer readable XML file entitled “GWP20240503322-sequence listing”, which was created on Jul. 2, 2024, with a file size of about 11,018 bytes, contains the sequence listing for this application, has been filed with this application, and is hereby incorporated by reference in its entirety.

### TECHNICAL FIELD

The present disclosure belongs to the technical field of molecular markers, and in particular relates to an adenosine triphosphatase sarcoplasmic/endoplasmic reticulum Ca²⁺ transporting 2 (ATP2A2) gene-based molecular marker for identifying chicken feed efficiency trait, and an identification method and use thereof.

### BACKGROUND

Feed efficiency traits are one of the most important economic traits in livestock production. A feed conversion ratio is an important index to measure feed efficiency. Residual feed intake (RFI) refers to a difference between an actual feed intake and an expected feed intake to maintain the growth needs for animals. RFI is an important index to measure feed conversion ratio. The lower the RFI, the higher the feed conversion ratio. RFI is a medium-to-high genetic trait independent of important growth traits such as an animal body weight and can improve feed efficiency without affecting the growth, fattening, and reproductive performance of livestock. There are many factors affecting RFI, and genetic factors are one of the most important factors affecting the RFI of livestock.

Adenosine triphosphatase sarcoplasmic/endoplasmic reticulum Ca²⁺ transporting 2 (ATP2A2) is one of the members of the ATP2A gene family. De et al. reported that, among mRNA-miRNAs for regulating RFI in Nelore cattle, differentially expressed genes are related to biological processes in animals such as lipid and fatty acid metabolism, energy and growth, xenobiotic metabolism, and oxidative stress, indicating that the ATP2A2 gene is up-regulated in a high feed efficiency group (De Oliveira P S N, Coutinho L L, Tizioto P C, et al. Anintegrative transcriptome analysis indicates regulatory mRNA-miRNAnetworksfor residual feed intake in Nelore cattle [J].Sci Rep, 2018, 8 (1): 17072-17084). Zhou et al. conducted a transcriptome study on pectoral muscles of broilers in high and low feed efficiency groups. Study results showed that an expression level of the differentially expressed gene ATP2A2 in pectoral muscles of broilers in a high feed efficiency group is higher than an expression level of the differentially expressed gene ATP2A2 in pectoral muscles of broilers in a low feed efficiency group, revealing that the ATP2A2 gene can maintain the energy required for the growth and development of an animal by increasing a Ca²⁺ concentration in the animal (Zhou N, Lee W R, AbashtB.Messenger RNAsequencing and pathway analysis provide novel insights into the biological basis of chickens' feed efficiency [J]. BMC Genomics, 2015, 16 (1): 195-215).

Therefore, the ATP2A2 gene may be an important candidate gene affecting chicken feed efficiency. In order to accurately select a genotype suitable for growth and development and thus provide data for early selective breeding, based on the above content, the present disclosure provides an ATP2A2 gene-based molecular marker for identifying a chicken feed efficiency trait, and an identification method and use thereof.

### SUMMARY

An objective of the present disclosure is to provide an ATP2A2 gene-based molecular marker for identifying a chicken feed efficiency trait, and an identification method and use thereof. Compared with the prior art, the present disclosure develops a single nucleotide polymorphism (SNP) molecular marker for a candidate gene (the ATP2A2 gene) related to the chicken feed efficiency trait to solve the problem of slow progress in conventional phenotypic selective breeding and allow the early identification of a feed efficiency trait.

The present disclosure adopts the following technical solutions to achieve the above objective:

An ATP2A2 gene-based molecular marker for identifying a chicken feed efficiency trait is provided, where the molecular marker is located in a promoter region of an ATP2A2 gene and has a nucleotide sequence shown in SEQ ID NO: 1, and a base at locus 926 in the nucleotide sequence is T or G.

A use of the ATP2A2 gene-based molecular marker for identifying a chicken feed efficiency trait in identification of a chicken feed efficiency trait is provided.

A method for identifying a chicken feed efficiency trait using the molecular marker is provided, including the following steps:
(1) extracting total DNA from venous blood of a chicken wing;
(2) designing specific amplification primers with a sequence including a locus at which the molecular marker is located and upstream and downstream bases thereof as a target sequence, and using the specific amplification primers to conduct PCR amplification with the total DNA as a template to obtain an amplification product;
(3) genotyping and sequencing the amplification product to obtain a molecular marker type of a chicken to be tested; and
(4) determining the chicken feed efficiency trait based on the molecular marker type.

As a further optimized solution of the present disclosure, sequences of the specific amplification primers are as follows:

SEQ ID NO: 2:
forward primer:
TTTAGCAGCGATGGATGT;
and
SEQ ID NO: 3:
reverse primer:
GCGAGGAGGTATGAACTG.

As a further optimized solution of the present disclosure, a method of the genotyping is as follows: cleaving the amplification product with a TatI restriction endonuclease to obtain a cleavage product, detecting the cleavage product by agarose gel electrophoresis, and allowing the genotyping according to an electrophoresis pattern, where if the cleavage product includes:
1 band, then the chicken feed efficiency trait is of a GG genotype;
2 bands, then a TT genotype; and
3 bands, then the chicken feed efficiency trait is of a TG genotype.

As a further optimized solution of the present disclosure, if the molecular marker type of the chicken to be tested is the GG genotype, the chicken feed efficiency trait is at a high level; if the molecular marker type of the chicken to be tested is the TT genotype, the chicken feed efficiency trait is at a low level; and if the molecular marker type of the chicken to be tested is the TG genotype, the chicken feed efficiency trait is at a medium level.

As a further optimized solution of the present disclosure, the cleavage product is detected by agarose gel electrophoresis with a concentration of 2.0% or more.

The present disclosure has the following beneficial effects:

In the present disclosure, by identifying a genotype of the molecular marker in a chicken genome and then selecting the chicken feed efficiency trait according to the genotype, a breeding method for early selection of the chicken feed efficiency trait is established. The method is simple, rapid, and low-cost, does not require special instruments, and is suitable for the needs of marker-assisted selection experiments.

### BRIEF DESCRIPTION OF THE DRAWINGS

FIG. 1 is an agarose gel electrophoresis pattern of PCR amplification products of some samples;

FIG. 2 is an agarose gel electrophoresis pattern of cleavage products resulting from enzyme cleavage of PCR amplification products of some samples; and

FIG. 3 shows sequencing results of genotype verification for locus 926 (locus 926 in SEQ ID NO: 1) in a promoter region of the ATP2A2 gene in chicken.

### DETAILED DESCRIPTION OF THE EMBODIMENTS

The present application will be described in further detail below with reference to the accompanying drawings. It should be pointed out herein that the following specific implementations are only intended to further explain the present application, rather than to be construed as limiting the protection scope of the present application. Those skilled in the art may make non-essential improvements and adjustments to the present application based on the above content.

### 1. Materials

All methods used in this embodiment are conventional methods known to those skilled in the art, unless otherwise specified. All materials and reagents used in this embodiment are commercially available, unless otherwise specified.

### 2. Methods

### 2.1 Primer Design

DNA sequence corresponding to a promoter of the ATP2A2 gene was found from a chicken genome database, and specific amplification primers were designed with a partial DNA sequence of a promoter region of the ATP2A2 gene (a sequence including a locus at which the polymorphic molecular marker of the present disclosure was located and upstream and downstream bases thereof) as a template. The partial DNA sequence of the promoter region of the ATP2A2 gene is shown in SEQ ID NO: 1. Sequences of the specific amplification primers were as follows:

SEQ ID NO: 2:
forward primer:
TTTAGCAGCGATGGATGT;
and
SEQ ID NO: 3:
reverse primer:
GCGAGGAGGTATGAACTG.

The amplified region of the primers had a length of 391 bp and a sequence shown in SEQ ID NO: 4, and included a molecular marker with a T/C mutation at locus 926 and a sequence shown in SEQ ID NO: 1.

2.2 Extraction of Total DNA from Blood

468 local roosters (Wannan three-yellow chicken) were selected, venous blood was collected from wings, and total DNA was extracted from the venous blood. The total DNA in a venous blood sample of a chicken wing was extracted using a blood DNA extraction kit produced by TIANGEN, and extraction steps were carried out according to instructions of the kit.

### 2.3 PCR Amplification

The synthesized sequencing-specific primers were used to conduct the PCR amplification of the target fragment of the ATP2A2 gene with the Mix produced by Yeasen Biotechnology (Shanghai) Co., Ltd., and a PCR amplification system is shown in Table 1:

TABLE 1
PCR amplification system

| Component | Volume |
|---|---|
| DNA template | 0.8 μL |
| Forward primer | 0.1 μL |
| Reveres primer | 0.1 μL |
| Mix | 7 μL |
| ddH₂O | 7 μL |
| Total | 15 μL |

PCR conditions were as follows: pre-denaturation at 95° C. for 5 min; step 1: denaturation at 95° C. for 45 s; step 2: annealing at 53° C. to 54° C. for 45 s (a temperature of the annealing was set according to the primers); step 3: extension at 72° C. for 30 s, where steps 2 and 3 were cycled 31 times, with a total of 32 cycles; and extension at 72° C. for 10 min.

### 2.4 Detection and Sequencing of PCR Amplification Products

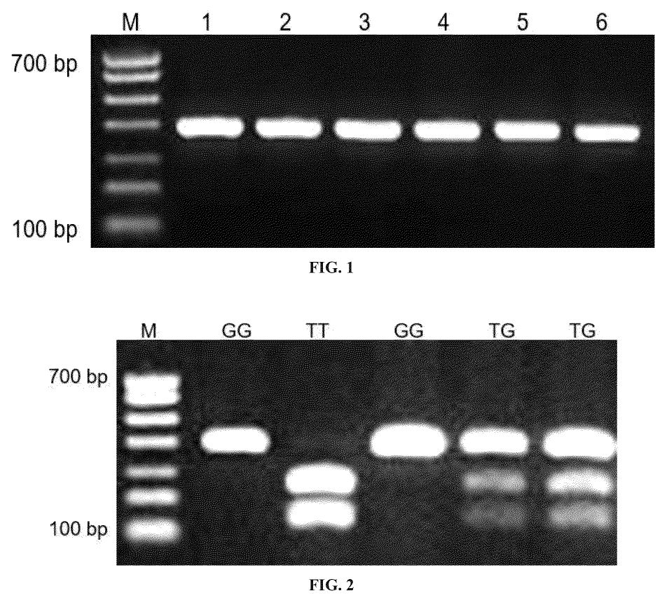

A PCR amplification product was detected by agarose gel electrophoresis with a mass ratio of 2%, and results are shown in FIG. 1. After imaging on a gel imager, a band with a length of approximately 391 bp was obtained, and this length was consistent with the predicted length, indicating that the target fragment was obtained. The PCR product was sent to Beijing Tsingke Biotechnology Co., Ltd. (Nanjing) for sequencing, and a sequence of the PCR product was shown in SEQ ID NO: 4, which was consistent with the predicted result.

### 2.5 Genotyping

2.5.1 An enzyme cleavage system shown in Table 2 was prepared. The PCR amplification product was cleaved with a Tat I restriction endonuclease of Thermo Fisher Scientific in a 65° C. water bath for 2 h.

TABLE 2
Enzyme cleavage system

| Component | Volume |
|---|---|
| PCR amplification product | 2 μL |
| Tat I | 1 μL (2 U/μL) |
| buffer | 1 μL |
| ddH₂O | 6 μL |
| Total | 10 μL |

2.5.2 The cleavage product was detected by low-voltage agarose gel electrophoresis with a mass ratio of 2%, and results are shown in FIG. 2 (partial results). If the cleavage product: included 1 band, it indicated a GG genotype; included 2 bands, it indicated a TT genotype; and included 3 bands, it indicated a TG genotype.

### 2.6 Enzyme Cleavage Sequencing Verification

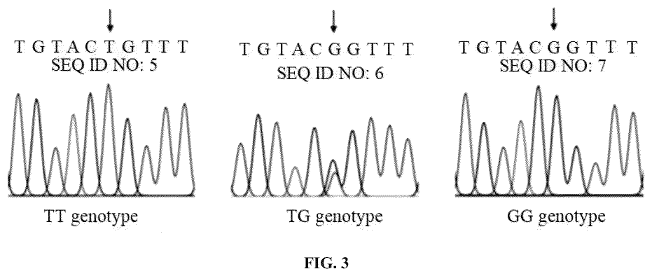

According to the agarose gel electrophoresis pattern of enzyme cleavage-based genotyping, the three genotypes of TT, TG, and GG were obtained. One individual was selected from each of the three genotypes for sequence alignment, and sequence alignment results are shown in FIG. 3. In the sequencing results, T was mutated into C, and the arrow marked the mutation locus, which was consistent with the enzyme cleavage-based genotyping result.

### 2.7 Effect Verification

In order to determine the correlation between the T/G polymorphism at locus 926 in the promoter region of the ATP2A2 gene and an important phenotypic trait of chicken, 468 Wannan three-yellow chickens in the section 2.2 were taken as experimental materials, and the average daily feed intake (ADFI), average daily gain (ADG), metabolic body weight gain (MBW^{0.75}), feed conversion ratio (FCR), and RFI of 56-98 day-old chicken were counted. The 468 Wannan three-yellow chickens were genotyped by the genotyping method in the section 2.5, and results are shown in Table 3.

TABLE 3
Genotyping results of individuals with different phenotypes

| | Genotype frequency | | | Gene frequency | | Chi-square test | |
|---|---|---|---|---|---|---|---|
| Locus | TT | TG | GG | T | G | χ² | P value |
| T-926G | 0.066 | 0.404 | 0.530 | 0.268 | 0.732 | 0.391 | 0.822 |

Conclusion: Results of the chi-square test showed that genotypes of the experimental chicken population were in Hardy-Weinberg equilibrium (P>0.05).

### 2.8 Statistical Analysis

The correlation between the three genotypes and the chicken feed efficiency trait was analyzed by the least-squares analysis method in the SAS9.4 software, and analysis results of the correlation between the different genotypes and the trait are shown in Table 4.

TABLE 4
Analysis of the correlation between the chicken ATP2A2
genotypes and the chicken feed efficiency trait

| | Genotype | | |
|---|---|---|---|
| Trait | TT (n = 31) | TG (n = 189) | GG (n = 248) |
| ADFI (g/d) | 42.18 ± 3.65 | 41.82 ± 3.42 | 41.19 ± 3.41 |
| ADG (g/d) | 13.47 ± 1.65 | 13.39 ± 1.82 | 13.57 ± 1.67 |
| MBW^{0.75} | 140.52 ± 7.79 | 141.72 ± 8.33 | 141.07 ± 9.18 |
| FCR (g/g) | 3.14 ± 0.37 | 3.16 ± 0.35 | 3.08 ± 0.23 |
| RFI (g/d) | 0.56 ± 0.80^{a} | 0.05 ± 0.85^{b} | −0.20 ± 0.92^{b} |

Note:
Different low case letters in the same row indicate significant differences (P < 0.05).

Conclusion: It can be seen from the comparison of feed efficiency traits of individuals with different genotypes in Table 4 that, in terms of the RFI, GG, and TG individuals are significantly lower than TT individuals (P<0.05). Since the RFI is a negative selection trait, it can be known that individuals with the GG genotype have the highest feed conversion trait, individuals with the TG genotype have the medium feed conversion trait, and individuals with the TT genotype have the lowest feed conversion trait.

The above are merely several embodiments of the present disclosure. Although the descriptions of these embodiments are specific and detailed, these embodiments should not be construed as limiting the patent scope of the present disclosure. It should be noted that those of ordinary skill in the art can further make several variations and improvements without departing from the concept of the present disclosure, and these variations and improvements all fall within the protection scope of the present disclosure.

1. (canceled)

2. A method for identifying a chicken feed efficiency trait using a molecular marker, comprising the following steps:
(1) extracting total DNA from venous blood of a chicken wing;
(2) designing specific amplification primers with a sequence comprising a locus at which the molecular marker is located and upstream and downstream bases thereof as a target sequence, and using the specific amplification primers to conduct PCR amplification with the total DNA as a template to obtain an amplification product;
(3) genotyping and sequencing the amplification product to obtain a molecular marker type of a chicken to be tested; and
(4) determining the chicken feed efficiency trait based on the molecular marker type; wherein
the molecular marker is an adenosine triphosphatase sarcoplasmic/endoplasmic reticulum Ca2+ transporting 2 (ATP2A2) gene-based molecular marker and has the sequence set forth in SEQ ID NO: 1; and wherein
if the molecular marker type of the chicken to be tested is a GG genotype, the chicken feed efficiency trait is at a high level;
if the molecular marker type of the chicken to be tested is a TT genotype, the chicken feed efficiency trait is at a low level;
if the molecular marker type of the chicken to be tested is a TG genotype, the chicken feed efficiency trait is at a medium level; and
the chicken to be tested is a Wannan three-yellow chicken, and the chicken feed efficiency trait is determined according to residual feed intake (RFI).

3. The method for identifying a chicken feed efficiency trait using the molecular marker according to claim 2, wherein sequences of the specific amplification primers are as follows:
SEQ ID NO: 2:
forward primer:
TTTAGCAGCGATGGATGT;
and
SEQ ID NO: 3:
reverse primer:
GCGAGGAGGTATGAACTG.

4. The method for identifying a chicken feed efficiency trait using the molecular marker according to claim 3, wherein a method of the genotyping is as follows: cleaving the amplification product with a TatI restriction endonuclease to obtain a cleavage product, detecting the cleavage product by agarose gel electrophoresis, and allowing the genotyping according to an electrophoresis pattern, wherein if the cleavage product comprises:
1 band, then the chicken feed efficiency trait is of a GG genotype;
2 bands, then the chicken feed efficiency trait is of a TT genotype; and
3 bands, then the chicken feed efficiency trait is of a TG genotype.

5. The method for identifying a chicken feed efficiency trait using the molecular marker according to claim 4, wherein the cleavage product is detected by agarose gel electrophoresis with a concentration of 2.0% or more.


SEQUENCE LISTING (7 sequences)

SEQ ID NO: 1 - DNA, 2000 bp, Gallus sp.
  source 1..2000: mol_type = genomic DNA; note = molecular marker located in a promoter region of an ATP2A2 gene
tctgttttcaatggataagagagaaacaaggaaagtggagctgtcaagctgtagcagaactagtgaatcctagagaatgccaagagttcaccaagattaggtagaatgcaaactctaccttcctagcaacatttctgcctgttaaccagtcggactactttttttttatacactgccaacctctaattgcacatgccatggttctggtatgcctaaatacttttctgtaagcctttttggaaacaagctcaaaaaggaaaccaataatgacatacagagtacataacagaaggaaataactatgaaacatgacagttttggggtttgttttttttttttttgttacagttagagcatatctacatgtgtgcagtgtgatgcagtgatccgagaagcaacgtaactgtattcaagtaacatttgggcacacagactaacctgtctaataataaaggataaaattaatatgggcatggagtaactaattctcgtacctaaactgggtcatcagatctaactcatgattttttgttcaggactgtcccgtgaagcgcagacatgctgtcaagaagcccttacctggacactggctaattacacaggaatctcttctggggcagctgcacttttgctagaaaggatgcagggcaacacagcacctaggatgccatattttatatcgctgtcccctctcctcacttcctttgccagtacagaggtaagattaagatattttccttccagatcagtttctcacagtgtgatactgtgggtggtttttagcagcgatggatgtgcagcttgtatgtaggatctgcatttatgtaagccagcacacatgaacagatcagctcttgtatttgcacatgcataagagctggttgaaaccaagtgcttttaagggaatttatactgtgctctgtactgtttgctattgctggtgacattgctggtgtcactgatgtcagacggtgctgatctaacagctcatactgcattcattgaagtctctgtgaattaaggggtaggatggctaaatcagtctgttttcagcattctgtgtctgtttttgagtctctgccacttacggagatgctgcgctgagggaagctgctatctcacaccacgcacactgctgaaatcacactgtgcagttcatacctcctcgcgttatcaccgtgcaagccagggctgagtgtgcgtgtcactgttatgctggcaggggcagcgtgcggcgcagcgggaattggggaagtcgtgtgaaagacacggctgtgccagcacaggccccgcacagcccagcgggacctctggggaacgaacccctctggtgccggcagggaggcacgtagccgcgcttcctatcctcgcccctggcggatccgtggcggggtgaggctcctgtcctcacagtacagcccctcaactcaccctttgcctgttttttgtcacgactgaggtggcggcagcccagccctcgctggccgtttacgtgaggttcctgtcacgactgaggtggcggtagccctaccctcactgtttggccgtttatgtgaggttcctgtcacgacagagccgggcagccccacacgcgcgctccaggctcgcattcctgtcaccgagcgcgctcccctcagccggggcagccccgcttccctctgccactctgcgcgggttgaagccgggacagagggcggccacagcggttcactccgggccaggggggcgggaaagcggccaccccacctgcaggcctctcccacggcggagcggaagcgctcgaccggccacacgaggcctgcatcaccctccgcctcctcctcctccacccctcagccgagggcgggcgaccgcgctgcgcaggaccgcgcctcagcgccgtggggcgtggccccgctctgttccgccccctcctctgggtcggccaatcagcggcgtccacatgccgcggcggtgagtggggcccccggcggcccagctcccgtaagttacattagagc

SEQ ID NO: 2 - DNA, 18 bp, synthetic construct
  source 1..18: mol_type = other DNA; note = forward primer for specific amplification
tttagcagcgatggatgt

SEQ ID NO: 3 - DNA, 18 bp, synthetic construct
  source 1..18: mol_type = other DNA; note = reverse primer for specific amplification
gcgaggaggtatgaactg

SEQ ID NO: 4 - DNA, 391 bp, Gallus sp.
  source 1..391: mol_type = genomic DNA; note = sequence of amplified region for the primers
tttagcagcgatggatgtgcagcttgtatgtaggatctgcatttatgtaagccagcacacatgaacagatcagctcttgtatttgcacatgcataagagctggttgaaaccaagtgcttttaagggaatttatactgtgctctgtactgtttgctattgctggtgacattgctggtgtcactgatgtcagacggtgctgatctaacagctcatactgcattcattgaagtctctgtgaattaaggggtaggatggctaaatcagtctgttttcagcattctgtgtctgtttttgagtctctgccacttacggagatgctgcgctgagggaagctgctatctcacaccacgcacactgctgaaatcacactgtgcagttcatacctcctcgc

SEQ ID NO: 5 - DNA, 10 bp, Gallus sp.
  source 1..10: mol_type = genomic DNA; note = sequencing result of genotype verification for locus 926
tgtactgttt

SEQ ID NO: 6 - DNA, 10 bp, Gallus sp.
  source 1..10: mol_type = genomic DNA; note = sequencing result of genotype verification for locus 926
tgtacggttt

SEQ ID NO: 7 - DNA, 10 bp, Gallus sp.
  source 1..10: mol_type = genomic DNA; note = sequencing result of genotype verification for locus 926
tgtacggttt